(19) United States
(12) Patent Application Publication
(10) Pub. No.: US 2011/0054233 A1
(43) Pub. Date: Mar. 3, 2011

(54) METHOD FOR OLIGOMERIZATION OF ETHYLENE AND REACTOR SYSTEM THEREFOR

(76) Inventors: Fuad Mousa, Riyadh (SA); Mohammed Al-Hazmi, Riyadh (SA); Abdullah Al-Thauyyan, Riyadh (SA); Mohammed Al-Musned, Riyadh (SA); Wolfgang Müller, Munich (DE); Peter M. Fritz, Unterhaching (DE); Heinz Bölt, Wolfratshausen (DE); Anton Wellenhofer, Munich (DE); Andreas Meiswinkel, Munich (DE); Carsten Taube, Ebersberg (DE); Richard Schneider, Uffing (DE); Florian Winkler, Munich (DE); Helmut Fritz, Munich (DE); Karl-Heinz Hofmann, Germering (DE); Hans-Jörg Zander, Munich (DE); Peter Ulbrich, Pullach (DE); Jan Segatz, Hohenschaftlarn (DE)

(21) Appl. No.: 12/735,267

(22) PCT Filed: Dec. 18, 2008
(86) PCT No.: PCT/EP2008/010802

§ 371 Date: Nov. 5, 2010

(30) Foreign Application Priority Data
Dec. 27, 2007 (EP) 07025109.5

(51) Int. Cl. C07C 2/08; B01J 19/00

(52) U.S. Cl. 585/522; 585/520; 585/523; 422/131

(57) ABSTRACT

The present invention relates to a method and a reactor system, for the oligomerization of ethylene, comprising oligomerizing ethylene in a reactor in the presence of a solvent and a catalyst composition to produce a liquid product stream comprising linear alpha-olefins, solvent and catalyst composition, and deactivating and extracting the catalyst composition in said liquid product stream by mixing it with a polar phase in a dynamic mixing device having rotor and stator elements comprising concentric tool rings.

The present invention relates to a method for the oligomerisation of ethylene and a reactor system therefore.

Oligomerisation methods for preparing linear alpha-olefins by oligomerisation of ethylene are widely known. These methods are usually carried out in a reactor system wherein ethylene introduced is converted by a suitable catalyst composition in presence of a solvent to linear alpha-olefins. After oligomerisation in the reactor gaseous and liquid outlet streams are further processed. The liquid outlet stream from the reactor usually contains linear alpha-olefins, solvent and the still active catalyst composition. One essential feature of the respective method is the deactivation of the components of the catalyst composition and the extraction from the organic phase. Usually, deactivation and extraction is achieved by mixing the liquid reactor outlet with a polar phase, e.g. an aqueous caustic solution. In this regard, a fast and effective mixing of the organic phase with the polar phase is required. Additionally, the formation of very small droplets and a minimum residence time of outlet stream and polar phase in the mixer (until effective mixing has been achieved) has to be ensured in order to create sufficient phase transfer surface. If the deactivation is not achieved fast enough, undesired side reactions will take place. This leads to a down grading of the product purities, and undesired by-products may form corrosive components in the separation section.

In the prior art static mixers, jet nozzles or stirred tank mixers have been utilized for mixing the organic outlet phase and the polar phase. However, these mixing devices have been so far insufficient to fulfil all the above requirements and can be further improved.

It is therefore an object of the present invention to provide a method for the oligomerisation of ethylene which overcomes the drawbacks of the prior art. Especially a method shall be provided wherein after oligomerisation the catalyst composition may be easily and fast deactivated and extracted from the organic outlet stream of the oligomerisation reactor.

Additionally, it is an object to provide a reactor system for such an oligomerisation.

The first object is achieved by a method for the oligomerisation of ethylene, comprising the steps of:
(i) oligomerising ethylene in a reactor in the presence of a solvent and a catalyst composition;
(ii) discharging a catalyst composition containing outlet stream from the reactor;
(iii) deactivating and extracting the catalyst composition with a polar phase, wherein the outlet stream and the polar phase are mixed in a dynamic mixing device having rotor and stator elements comprising concentric tool rings.

Preferably the mixing device includes elements that are radially slotted and/or drilled, wherein the annular shearing gap between adjacent tool rings is from 0.1 to 5 mm;

Preferably, the polar phase is an aqueous caustic solution.

More preferably, the rotational speed of the dynamic mixer is from 2.5 to 40 m/s.

In one embodiment, the catalyst composition comprises a zirconium salt of organic acids and at least one organo aluminum compound.

Further, it is preferred, that the zirconium salt has the formula ZrCl₄₋ₘXₘ, wherein X=OCOR or OSO₃R′ with R and R′ being independently alkyl, alkene or phenyl, and wherein 0<m<4.

Additionally, it is preferably proposed that at least one aluminum compound has the general formula R¹ₙAl₃₋ₙ or Al₂Y₃R¹₃, wherein R¹ represents an alkyl group having from 1 to 20 carbon atoms, Y represents Cl, Br or I, n is any number within the range 1<n<2.

According to the invention is also a reactor system for the oligomerisation of ethylene, comprising a reactor having inlets and outlets for feeding and discharging ethylene, alpha-olefins, solvent and catalyst compositions to and from the reactor, the reactor being connected with a dynamic mixing device where a catalyst composition containing outlet stream is mixed with a polar phase for deactivation and extraction of the catalyst, the dynamic mixing device having rotor and stator elements comprising concentric tool rings that are radially slotted and/or drilled, wherein the annular shearing gap is between 0.1 and 5 mm.

Preferably the dynamic mixing device includes cutting devices for treating of high molecular weight linear alpha-olefins up-streams of the stator and rotor elements or integrated into an inlet zone of the dynamic mixing device or in a separate casing.

Surprisingly it was found that the use of the specific dynamic mixing device as disclosed above results in a method for the oligomerisation of ethylene wherein a fast and effective mixing of liquid reactor outlet streams containing the catalyst composition with a polar phase for deactivation and extraction can be achieved. Additionally, very small droplets are formed and a minimum residence time until effective mixing has been performed is achieved. Further, the deactivation utilizing the dynamic mixing device is fast enough, so that undesired side reactions will be avoided. Thus, no downgrading of product purities is obtained, and a formation of corrosive components in the separation section is avoided.

By adapting the mixing device geometry as desired, the droplet size can be adjusted to fulfil the required fast and effective deactivation and extraction. On the other hand, the formation of a stable emulsion can be avoided so that the separation of the organic phase from the polar phase by gravity separation in a decanter can still be achieved afterwards.

In detail, the performance of the dynamic mixing device can be optimized by adaptation of the mixer geometry, like the gaps and distances between rotor and stator elements and the applied rotational speed.

It has been found that mixer types in which mixing shall be achieved by turbulence are not suitable for the purposes of the present invention, but high shear forces are needed to fulfil the required highly efficient mixing task. Satisfying results were only achieved utilizing the dynamic mixer as disclosed above. High shear forces and low residence time can then be realized.

The droplet size achieved is around 10 μm with a narrow size distribution.

In a further embodiment, the mixing device may include cutting devices for treating of high molecular weight linear alpha-olefins upstreams of the mixing elements or integrated into an inlet zone of the dynamic mixing device or in a separate casing. This assists in avoiding deposition and plugging of the reactor system.

Further, dead zones within the dynamic mixing device can be significantly avoided which also adds to the benefits of the inventive method and reactor system.

The features disclosed in the foregoing description, or in the claims may, both separately and in any combination thereof, be material for realizing the invention in diverse forms thereof.

1. A method for the oligomerization of ethylene, comprising the steps of:
oligomerizing ethylene in a reactor in the presence of a solvent and a catalyst composition and forming a liquid product stream comprising linear alpha-olefins, solvent and the catalyst composition;
(ii) deactivating and extracting the catalyst composition in said liquid product stream by mixing it with a polar phase in a dynamic mixing device having rotor and stator elements comprising concentric tool rings, that are radially slotted and/or drilled, having an annular shearing gap is from 0.1 to 5 mm.

2. The method according to claim 1, wherein the polar phase is an aqueous caustic solution.

3. The method according to claim 1, wherein the rotational speed of the dynamic mixing device is from 2.5 to 40 m/s.

4. The method according to claim 2, wherein the catalyst composition comprises a zirconium salt of organic acids and at least one organoaluminum compound.

5. The method according to claim 4, wherein the zirconium salt has the formula ZrCl₄₋ₘXₘ, wherein X=OCOR or OSO₃R′ with R and R′ being independently alkyl, alkene or phenyl, and wherein 0≦m≦4.

6. The method according to claim 4, wherein the at least one organoaluminum compound has the general formula R¹ₙAl₃₋ₙ or Al₂Y₃R¹₃, wherein R¹ represents an alkyl group having from 1 to 20 carbon atoms, Y represents Cl, Br or I, n is any number within the range 1≦n≦2.

7. A reactor system for the oligomerization of ethylene, comprising a reactor having inlets and outlets for feeding and discharging ethylene, alpha-olefins, solvent and catalyst composition to and from the reactor, the reactor product outlet being connected with a dynamic mixing device wherein the reactor product outlet stream is mixed with a polar phase by a dynamic mixing device having rotor and stator elements comprising concentric tool rings, that are radially slotted and/or drilled, having an annular shearing gap is from 0.1 to 5 mm.

8. The reactor system according to claim 7, wherein the dynamic mixing device has cutting devices for treating high molecular weight linear alpha-olefins upstreams of the stator and rotor elements.

9. The reactor system of claim 7, wherein the dynamic mixing device has a cutting device for treating high molecular weight linear alpha-olefins in an inlet zone of the dynamic mixing device.

10. The method according to claim 4, wherein the rotational speed of the dynamic mixing device is from 2.5 to 40 m/s.

11. The method according to claim 6, wherein the rotational speed of the dynamic mixing device is from 2.5 to 40 m/s.
